(19) United States
(12) Patent Application Publication
(10) Pub. No.: US 2004/0253319 A1
(43) Pub. Date: Dec. 16, 2004

(54) Pharmaceutical compositions and method for alleviating side-effects of estrogen replacement therapy

(76) Inventors: Shrirang Netke, San Bruno, CA (US); Aleksandra Niedzwiecki, San Jose, CA (US); Matthias Rath, Almelo (NL); Vadim Ivanov, Castro Valley, CA (US)

Correspondence Address: KENYON & KENYON, ONE BROADWAY, NEW YORK, NY, 10004, US

(21) Appl. No.: 10/460,023

(22) Filed: Jun. 11, 2003

(51) Int. Cl. A61K 31/56; A61K 35/78; A61K 33/04; A61K 33/34; A61K 33/32; A61K 31/198; A61K 31/375

(52) U.S. Cl. 424/630; 424/639; 424/702; 424/729; 514/171; 514/562; 514/564; 514/474

(57) ABSTRACT

The present invention provides pharmaceutical compositions for alleviating pathological conditions in a post-menopausal woman, comprising lysine, proline, arginine, ascorbic acid, magnesium, green tea extract, N-acetyl-cysteine, selenium, copper, manganese and one pharmaceutical acceptable component selected from the group consisting of a carrier, a diluent, and an excipient, wherein the compositions contain 24-25 wt % lysine, 16-25 wt % ascorbic acid and 22-25 wt % green tea extract. A method of treatment using the pharmaceutical compositions are also disclosed.

### FIELD OF THE INVENTION

The present invention relates to pharmaceutical compositions and methods of alleviating side-effects of estrogen replacement therapy in post-menopausal women, particularly relating to cardiovascular and neoplastic diseases.

### SUMMARY OF THE INVENTION

The present invention provides pharmaceutical compositions for alleviating pathological conditions in a post-menopausal woman, comprising lysine, proline, arginine, ascorbic acid, magnesium, green tea extract, N-acetyl-cysteine, selenium, copper, manganese, wherein the compositions contain 24-25 wt % lysine, 16-25 wt % ascorbic acid and 22-25 wt % green tea extract. The present invention also provides a method of treatment using the pharmaceutical compositions.

### BACKGROUND OF THE INVENTION

Post-menopausal syndrome affects women who have entered into menopause. Common symptoms include osteoporosis, nausea, constipation, diarrhea, arthralgia, myalgia, hot flashes, sweating, psychological and emotional symptoms of fatigue, insomnia, irritability and nervousness. See, The Merck Manual, 1793 (16^{th} Ed. 1992). Estrogen replacement therapy has become a standard clinical remedy for post-menopausal syndromes in the United States and many other countries. The hormonal therapy renders certain advantages. For example, data support the ability of estrogens to limit the progression of osteoporotic bone loss. Some studies support a cardioprotective effect of estrogen by showing that post-menopausal estrogen-replacement therapy reduces both the incidence of coronary heart disease and mortality from cardiovascular disease (Stampfer, M. J. et al., N. Engl. J. Med. 325, 756-762 (1991)). Despite these reported beneficial effects, estrogen replacement therapy also suffers from undesirable side-effects. For example, the beneficial cardiovascular effects of estrogen replacement have not been confirmed in more recent studies. The mega analysis of recent clinical trials indicates that hormone replacement therapy does not provide cardiovascular benefits as once thought, instead it is found to be potentially harmful (Waters et al. JAMA, vol. 288, pp. 2432-2440, 2002).

Among the numerous pathologies noted, two major side-effects of estrogen replacement therapy are of great long-term medical concern: a) cardiovascular abnormalities such as hypertension and atherosclerosis; and b) estrogen-dependent cancer, particularly breast cancer.

Estrogen replacement therapy is known to be associated with an increase in the incidence of cardiovascular diseases which include thrombo-embolitic, ischemic and hypertensive diseases. This increase in cardiovascular disease may relate to estrogen's potent ability to induce smooth muscle cells to proliferate, resulting in hypertension. Additionally, it may accelerate the progression of atherosclerosis. In the case of thrombo-embolitic and ischemic disease, increase hypertension, estorgen replacement therapy should be stopped immediately (Pschyrembel, Klinisches Worterbuch, 259 Edition).

Estrogen replacement therapy also associates with an increased incidence of neoplastic diseases, in particular, breast cancers. For example, the risk of breast cancer in women taking estrogen therapy is approximately 7% as compared with 2% in women not receiving estrogen therapy. Long-term use of estrogens and related hormones may lead to proliferation of cancer cells as well as promote the spread of cancer cells.

Current approaches in alleviating the estrogen therapy side-effects in post-menopausal women include the administration of 1) chemotherapy compounds such as tamoxifen, 2) anti-estrogen compounds such as weak androgens, or 3) progestins. Such combined therapy is not ideal. For example, androgens may still exert stimulatory effects on certain cancer cell populations in the uterus and they have contraproductive effects. Continuous administration of progestins may induce amenorrhea and cause regressions of endometrial growths. The chronic use of progestin may cause unpleasant central nervous system side-effects and can lead to infertility.

Several U.S. patents discloses dietary supplements generally applicable in post-menopausal women. For example, U.S. Pat. No. 5,514,382 discloses a daily supplement of vitamin C, manganese, magnesium bioflavonoids, and selenium. U.S. Pat. No. 5,569,459 discloses a supplement of phytoestrogen, magnesium, calcium, vitamin E, ginseng root powder and pantothenic salt. U.S. Pat. No. 5,654,011 discloses a supplement of phytoestrogen and vitamin. U.S. Pat. No. 5,998,401 discloses a class of substituted napththalene compounds. U.S. Pat. No. 6,359,017 discloses a supplement of phytoestrogen and phytoandrogen. U.S. Pat. No. 6,476,012 discloses analogs of estradiol, optionally with vitamin C. U.S. Pat. No. 6,479,545 discloses a supplement of fatty acid compounds, calcium, vitamin C, and folic acid. All of the disclosed supplements could be improved to alleviate specific side-effects of estrogen replacement therapy as well as their effectiveness.

European Patent Application 00115643.9 discloses a pharmaceutical composition generally applicable in degenerative diseases associated with degradation of the extracellular matrix such as atherosclerosis, cancers and other related diseases. The composition includes ascorbate, fibrinolytic inhibitors and other trace elements.

There is a long felt need to provide a safe and effective pharmaceutical composition and method for alleviating the side-effects, primarily those of cardiovascular and neoplastic abnormalities, associated with the hormonal replacement therapy using synthetic estrogen and progestin drugs.

### OBJECT AND SUMMARY OF THE INVENTION

It is an object of the present invention to provide pharmaceutical compositions useful for alleviating pathological conditions of post-menopausal symptoms in women receiving estrogen therapy. The pathological conditions include symptoms due to the adverse cardiovascular effects (e.g., hypertension and atherosclerosis) and adverse neoplastic effects (e.g., breast cancer) in these women as a result of estrogen therapy.

Accordingly, the present invention provides pharmaceutical compositions for alleviating pathological conditions in a post-menopausal woman, comprising lysine, proline, arginine, ascorbic acid, magnesium, green tea extract, N-acetyl-cysteine, selenium, copper, manganese and one component selected from the group consisting of a pharmaceutically acceptable carrier, diluent, and excipient, wherein the compositions contain 24-25 wt % lysine, 16-25 wt % ascorbic acid and 22-25 wt % green tea extract.

Optionally, the pharmaceutical compositions further comprise an estrogen compound selected from the group consisting of ethynyl estrogen, mestranol, estradiol, ethinyl estradiol, estriol, norethisterone, lynestrenol, ethynodiol, dienoestrol, biperazine estrone sulfate, and phytoestrogen.

Optionally, the pharmaceutical compositions further comprise a progestin compound selected from the group consisting of medroxyprogesterone, norethylnodrel, and norethindrone.

The present invention provides a pharmaceutical composition comprising lysine 750 mg-15 gram, proline 500 mg-10 gram, arginine 400 mg-8 gram, ascorbic acid 500 mg-10 gram, magnesium 40 mg-750 mg, green tea extract 750 mg-15 gram, N-acetyl-cysteine 150 mg-2 gram, selenium 20-700 mcg, copper 1.5 mg-20 mg, and manganese 0.8 mg-15 mg, wherein the composition contains 24-25 wt % lysine, 16-25 wt % ascorbic acid and 22-25 wt % green tea extract.

Preferably, the present invention provides a pharmaceutical composition comprising lysine 1 gram-5.5 gram, proline 750 mg-4 gram, arginine 500 mg-3 gram, ascorbic acid 710 mg-4 gram, magnesium 50 mg-300 mg, green tea extract 1 gram-5 gram, N-acetyl-cysteine 200 mg-1 gram, selenium 30-400 mcg, copper 2 mg-10 mg, and manganese 1 mg-8 mg, wherein the composition contains 24-25 wt % lysine, 16-25 wt % ascorbic acid and 22-25 wt % green tea extract.

More preferably, the present invention provides a pharmaceutical composition comprising lysine 1 gram, proline 750 mg, arginine 500 mg, ascorbic acid 710 mg, magnesium 50 mg, green tea extract 1 gram, N-acetyl-cysteine 200 mg, selenium 30 mcg, copper 2 mg, and manganese 1 mg, wherein the composition contains 24-25 wt % lysine, 16-25 wt % ascorbic acid and 22-25 wt % green tea extract.

Preferably, the pharmaceutical compositions are in oral or parenteral form. More preferably, the oral form is a tablet or a capsule.

The present invention provides a method for alleviating pathological conditions in a post-menopausal woman, comprising the step of administering to the woman in need of treatment an effective amount of the pharmaceutical composition comprising lysine, proline, arginine, ascorbic acid, magnesium, green tea extract, N-acetyl-cysteine, selenium, copper, manganese and one component selected from the group consisting of pharmaceutically acceptable carrier, diluent, and excipient, wherein the composition contains 24-25 wt % lysine, 16-25 wt % ascorbic acid and 22-25 wt % green tea extract. Optionally, the composition comprises an estrogen compound and/or a progestin compound.

The present invention provides a method for alleviating pathological conditions in a post-menopausal woman, comprising the step of administering to the woman in need of treatment an effective amount of the pharmaceutical composition. Typically, it is recommended for a daily dosage of 10-208 mg/kg lysine, 7-139 mg/kg proline, 5-111 mg/kg arginine, 7-139 mg/kg ascorbic acid, 0.5-10 mg/kg magnesium, 10-208 mg/kg green tea extract, 2-28 mg/kg N-acetyl-cysteine, 0.0003-0.01 mg/kg selenium, 0.02-0.3 mg/kg copper, 0.01-0.2 mg/kg manganese, wherein the composition contains 24-25 wt % lysine, 16-25 wt % ascorbic acid and 22-25 wt % green tea extract.

Preferably, a daily dosage of the pharmaceutical composition includes: 13-70 mg/kg lysine, 10-56 mg/kg proline, 7-42 mg/kg arginine, 9.8-4 mg/kg ascorbic acid, 0.7-4.2 mg/kg magnesium, 13-70 mg/kg green tea extract, 3-14 mg/kg N-acetyl-cysteine, 0.0004-0.006 mg/kg selenium, 0.03-0.15 mg/kg copper, 0.01-0.1 mg/kg manganese, wherein the composition contains 24-25 wt % lysine, 16-25 wt % ascorbic acid and 22-25 wt % green tea extract.

More preferably, a daily dosage of 13 mg/kg lysine, 10 mg/kg proline, 7 mg/kg arginine, 9.8 mg/kg ascorbic acid, 0.7 mg/kg magnesium, 13 mg/kg green tea extract, 3 mg/kg N-acetyl-cysteine, 0.0004 mg/kg selenium, 0.03 mg/kg copper, 0.01 mg/kg manganese, wherein the composition contains 24-25 wt % lysine, 16-25 wt % ascorbic acid and 22-25 wt % green tea extract.

Preferably, the pharmaceutical compositions may be administered orally, intravenously, or parenterally.

### BRIEF DESCRIPTION OF THE DRAWINGS

FIG. 1 is a graph which shows the effect of the composition (20 mcg/ml) of the present invention on [³H] thymidine incorporation in human smooth muscle cells. Thymidine incorporation is expressed as a percentage of the value for the control group (100%).

FIG. 2 is a graph shows the effect of the composition (20 mcg/ml) on blocking estrogen (100 nM) mediated smooth muscle cell invasion.

FIG. 3 is a graph which shows the effect of the composition (20 mcg/ml) on blocking estrogen (at 100 nM) mediated interleukin-6 release in human smooth muscle cells.

FIG. 4 is a graph which shows the effect of the composition (100 mcg/ml) on blocking estrogen (20-500 nM) mediated [³H]thymidine incorporation in human breast cancer cells (MCF-7 cells).

FIG. 5 is a graph which shows the effect of the composition (30 mcg/ml) on blocking phytoestrogen-mediated (25 μM) human breast cells (MCF-7) proliferation as reflected by [³H]thymidine incorporation in these cells.

FIG. 6 is a graph which shows the effect of the composition (100 mcg/ml) on blocking estrogen (10-100 nM)-mediated VEGF release in human breast cancer cells (MCF-7 cells).

### DETAILED DESCRIPTION OF THE INVENTION

As used herein, the term “alleviating” is used to mean reducing, inhibiting, attenuating or treating the syndromes common to post-menopausal women receiving estrogen therapy. “Syndromes of estrogen therapy” is a well-recognized term and refers predominately herein to cardiovascular and neoplastic problems in women receiving estrogen replacement therapy including hypertension, atheroscloerosis and breast cancer. The term “effective amount” means an amount of composition of the present invention which is capable of alleviating the symptoms of the various pathological conditions herein described. The term “pharmaceutically acceptable” in reference to carriers, diluents, and excipients means that they must be compatible with the other ingredients of the formulation, and not deleterious to the recipient thereof. “Wt %” refers to % of the ingredient as a proportion of the total weight of the composition; for example, 25 wt % of lysine indicates that 25% of the total weight of the composition is made up of lysine.

The amount of estradiol and progesterone used in hormonal therapy can vary widely. An exemplary dosage for estradiol is 0.2-0.5 mg. An exemplary dosage for progesterone is 50-100 mg.

The present invention provides compositions for treating pathological conditions associated with estrogen replacement therapy in a post-menopausal woman, comprising lysine, proline, arginine, ascorbic acid, magnesium, green tea extract, N-acetyl-cysteine, selenium, copper, and manganese, an optionally estrogen or progestin.

Although not wishing to be bound by theory, the compositions of the present invention are effective in inhibiting estrogen-induced smooth muscle cell proliferation and invasion. Because smooth muscle cell proliferation and invasion play a central role in narrowing the arteriole, the compositions regulate the blood pressure as well as development of atheroscloerotic plaques. It appears that the combined effect of ingredients such as lysine and proline may prevent severe connective tissue degradation which in turn may attenuate the process of proliferation and invasion. Additionally, green tea extract and vitamin C may also blunt the connective tissue degradation by virtue of their anti-oxidant property. Although the exact mechanism of action is not fully understood, it probably is achieved through the synergistic effects of the ingredients present in the compositions in counteracting the estrogen's effects of cardiovascular degradation and cancer development.

The method of treating estrogen and progesterone deficiency after menopause varies. This generally involves the administration of an orally active, injectable or transdermal preparation of estrogen and an oral or injectable form of progestin. Clinical studies have demonstrated that the optimum dosage for the formulation of this invention is 3 capsules per day, with each capsule containing 0.3-0.4 mg of estradiol and 50 or 100 g of micronized progestin.

According to the present invention, the pharmaceutical compositions are useful for alleviating the symptoms of post-menopausal symptoms in women who receive estrogen therapy.

Various forms of estrogen are commercially available. Estrogen includes, for example, ethynyl estrogen (0.01-0.03 mg/day), mestranol (0.05-0.15 mg/day), and conjugated estrogenic hormones such as Premarin RTM. (Wyeth-Ayerst; 0.3-2.5 mg/day). An exemplary estrogen is Premarin.

Once absorbed by the human body, estrogen is converted to estradiol (17β estradiol), which is the biologically active metabolite of estrogen. The daily dose of estrogen is about 375 mcg to 1.25 mg per day, which is equivalent to a daily dose of estradiol of 0.05 mg to 1 mg. Other functional equivalents of estrogen include ethinyl estradiol, ethynodiol, dienestrol, estradiol, and biperazine estrone sulfate.

Menopausal syndrome is associated with changes in the estrogen profile in the body with advancing age. There is evidence that foodstuffs high in phytoestrogens are a suitable alternative to synthetic hormones in this respect, producing alleviation of adverse clinical symptoms. Phytoestrogens are believed to function by restoring balance to estrogen metabolism.

A phytoestrogen is a plant-derived estrogen compound. There are 3 principal classes of phytoestrogens; namely, isoflavones, lignans, and coumestans. Exemplary phytoestrogens include Genistein (5,7-dihydroxy-3-(4-hydroxyphenyl)-4H-1-benzopyran-4-one) and Resveratrol (5-[(1E)-2-(4-hydroxyphenyl)ethenyl]-1,3-benzendiol). Phytoestrogen has been shown to bind to the estrogen receptor, albeit at a lower affinity, and mimic estrogen's biological effects. There are no established dosages for phytoestrogen replacement therapy; some clinical studies with Genistein suggest a daily dose of 20 mg to 600 mg.

The phytoestrogen in accordance with the invention may be obtained from a number of different sources. Preferably the phytoestrogens are extracted from a clover such as red clover or subterranean clover, or from soya which contain high levels of phytoestrogens. However, any source rich in phytoestrogens may be used instead, if desired.

Various forms of progestins are also commercially available. Progestins include, for example, medroxyprogesterone such as Provera RTM. (Upjohn; 2.5-10 mg/day), norethylnodrel (1.0-10.0 mg/day), and nomethindrone (0.5-2.0 mg/day). Exemplary progestins are norethylnodrel and norethindrone.

When referred to herein, estrogen compound may include estrogen, estradiol, ethinylestradiol, estriol, norethisterone, and lynestrenol.

Lysine may include lysine salts such as hydroxylysine and hydroxylysine salts. Typically, the L-lysine is administered in a daily dose of 10 to 208 mg/kg, preferably, 13 to 70 mg/kg and more preferably, 13 mg/kg. L-lysine may be administered orally in a dosage form once, twice or three times a day. For an average individual weighing 72 kg, the recommended total amount of lysine per daily administration is 750 mg to 15 grams, preferably, 1 gram to 5.5 gram and more preferably 1,000 mg.

Proline may include proline, proline salts, hydroxyproline and hydroxyproline salts. Typically, the L-proline is administered in a daily dose of 7 to 139 mg/kg, preferably, 10 to 56 mg/kg and more preferably, 10 mg/kg. L-proline may be administered orally in a dosage form once, twice or three times a day. For an average individual weighing 72 kg, the recommended total amount of proline per daily administration is 500 mg to 10 grams, preferably, 750 mg to 4 gram and more preferably 750 mg.

Arginine may include arginine and arginine salts thereof. Typically, the L-arginine is administered in a daily dose of 5 to 111 mg/kg, preferably, 7 to 42 mg/kg and more preferably, 7 mg/kg. L-arginine may be administered orally in a dosage form once, twice or three times a day. For an average individual weighing 72 kg, the recommended total amount of arginine per daily administration is 400 mg to 8 grams, preferably, 500 mg to 3 gram and more preferably 500 mg.

Ascorbic acid may include ascorbic acid, ascorbate salts and its derivatives thereof. As used herein, ascorbic acid and vitamin C are used interchangeably and include calcium ascorbate, magnesium ascorbate or ascorbyl palmitate. Typically, ascorbic acid is administered in a daily dose of 7 to 139 mg/kg, preferably, 9.8 to 4 mg/kg and more preferably, 9.8 mg/kg. Ascorbic acid may be administered orally in a dosage form once, twice or three times a day. For an average individual weighing 72 kg, the recommended total amount of ascorbic acid per daily administration is 500 mg to 10 grams, preferably, 710 mg to 4 gram and more preferably 710 mg.

The different compounds claimed in this application can be used together in the form of covalently bound compounds or as physical mixture or in any other combination.

While not wishing to be bound by any theory, it is believed that the present compositions may exert beneficial effects via their ability to inhibit degradation of extracellular cell matrix. Cardiovascular diseases related to estrogen replacement therapy may be attributed to the degradation of the extracellular matrix. Moreover, the metastasis of cancer may be attributed to estrogen's ability to weaken the extracellular matrix, via the activation of enzymes including plasmin and collagenases that degrade the connective tissue.

The present invention provides pharmaceutical compositions including estrogen, an ascorbate compound, proline, lysine, or any combination thereof Therefore, the present invention is not limited to estrogen, ascorbate, proline or lysine, but embodies any equivalent structures that may be used in accordance with the preferred uses of the present invention.

Green tea extract as used herein refers to polyphenolic compounds that are present in green tea. Polyphenolic compounds may be present as up to 30% dry weight in green tea. They include flavanols, flavandiols, flavonoids, and phenolic acids. Flavanols represent the most abundant polyphenols in green tea and are commonly known as catechins. The major catechins in green tea extract include: 1) (−)-epicatechin, 2) (−)-epicatechin-3-gallate, 3) (−)-epigallocatechin, and 4) (−)-epigallocatechin-3-gallate (EGCG). Among the catechins, EGCG is the major polyphenolic constitutent present in green tea. As used herein, green tea extract contains about 80% by weight polyphenols and is free of caffeine.

Green tea extract may be administered in a daily dose of 10 to 208 mg/kg, preferably, 13 to 70 mg/kg and more preferably, 13 mg/kg. Green tea extract may be administered orally in a dosage form once, twice or three times a day. For an average individual weighing 72 kg, the recommended total amount of green tea extract per daily administration is 750 mg to 15 grams, preferably 1 gram to 5 grams and more preferably 1 gram.

N-acetyl-cysteine may include cysteine or cystine (dimer of cysteine) and cysteine salts thereof. N-acetyl-cysteine may be administered in a daily dose of 2 to 28 mg/kg, preferably, 3 to 14 mg/kg and more preferably, 3 mg/kg. N-acetyl-cysteine may be administered orally in a dosage form once, twice or three times a day. For an average individual weighing 72 kg, the recommended total amount of N-acetyl-cysteine per daily administration is 150 mg to 2 grams, preferably 200 mg to 1 gram and more preferably 200 mg.

The present invention further provides minerals and/or trace element. Trace elements may help to catalyze the production of these macromolecules needed for connective tissues.

Magnesium may be administered in a daily dose of 0.5 to 10 mg/kg, preferably, 0.7 to 4.2 mg/kg and more preferably, 0.7 mg/kg. Magnesium may be administered orally in a dosage form once, twice or three times a day. For an average individual weighing 72 kg, the recommended total amount of magnesium per daily administration is 40 mg to 750 grams, preferably, 50 mg to 300 gram and more preferably 50 mg.

Selenium may be administered in a daily dose of 0.0003 to 0.01 mg/kg, preferably, 0.0004 to 0.006 mg/kg and more preferably, 0.0004 mg/kg. Selenium may be administered orally in a dosage form once, twice or three times a day. For an average individual weighing 72 kg, the recommended total amount of selenium per daily administration is 20 mcg to 700 mcg, preferably, 30 mcg to 400 mcg and more preferably 30 mcg.

Copper may be administered in a daily dose of 0.02 to 0.3 mg/kg, preferably, 0.03 to 0.15 mg/kg and more preferably, 0.03 mg/kg. Copper may be administered orally in a dosage form once, twice or three times a day. For an average individual weighing 72 kg, the recommended total amount of copper per daily administration is 1.5 mg to 20 mg, preferably 2 mg to 10 mg and more preferably 2 mg.

Manganese may be administered in a daily dose of 0.01 to 0.2 mg/kg, preferably 0.01 to 0.1 mg/kg, and more preferably 0.01 mg/kg. Manganese may be administered orally in a dosage form once, twice or three times a day. For an average individual weighing 72 kg, the recommended total amount of manganese per daily administration is 0.8 mg to 15 mg, preferably 1 mg to 8 mg and more preferably 1 mg.

According to the present invention, some ingredients of the composition are present at a high amount. Lysine is present between 24-25 wt % (compared to the total composition), preferably, at 24% wt %. Vitamin C is present between 16-25 wt % (compared to the total composition), preferably at 17%. Green tea extract is present between 22-25 wt % (compared to the total composition), preferably between 22-24 wt %, more preferably at 24 wt %.

While not wishing to be bound by theory, it is believed that high proportions of these ingredients (i.e., 24-25 wt % lysine, 16-25 wt % ascorbic acid, and 22-25 wt % green tea extract), either independently or synergistically act to counteract the side-effects of estrogen replacement therapy.

The compositions of the present invention are useful in treating or inhibiting cardiovascular diseases which are characterized by excessive smooth muscle cell proliferation (smooth muscle cell hyperproliferation). The compositions are particularly useful in treating hypertension and atherosclerosis which frequently arise due to smooth muscle cell hyperproliferation in women receiving estrogen replacement therapy.

The compositions of the present invention are also useful in treating or inhibting neoplastic diseases such as breast cancer which is characterized by cancer cell proliferation and metastatsis.

The present invention also provides a method of treating post-menopausal syndrome in women comprising the step of administering to a woman an effective amount of the compositions of the present invention. The treatment is particularly useful for treating cardiovascular abnormalities (e.g., hypertension and atherosclerosis) and neoplasm (e.g., breast cancer) because the patient will receive the benefits of the estrogen therapy while the compositions of the present invention inhibit the undesirable side-effects of estrogen. The treatment may also be beneficial for the combined hormonal therapy (i.e., estrogen and progestin).

The dosage requirements vary with the route of administration, the severity of the symptoms presented and the particular subject being treated. A recommended daily dosage of the composition would be mg/kg administered orally. It is recommended for a daily dosage of 10-208 mg/kg lysine, 7-139 mg/kg proline, 5-111 mg/kg arginine, 7-139 mg/kg ascorbic acid, 0.5-10 mg/kg magnesium, 10-208 mg/kg green tea extract, 2-28 mg/kg N-acetyl-cysteine, 0.0003-0.01 mg/kg selenium, 0.02-0.3 mg/kg copper, 0.01-0.2 mg/kg manganese. Preferably, the daily dosage is: 13-70 mg/kg lysine, 10-56 mg/kg proline, 7-42 mg/kg arginine, 9.8-4 mg/kg ascorbic acid, 0.7-4.2 mg/kg magnesium, 13-70 mg/kg green tea extract, 3-14 mg/kg N-acetyl-cysteine, 0.0004-0.006 mg/kg selenium, 0.03-0.15 mg/kg copper, 0.01-0.1 mg/kg manganese. More preferably, a daily dosage is: 13 mg/kg lysine, 10 mg/kg proline, 7 mg/kg arginine, 56 mg/kg ascorbic acid, 0.7 mg/kg magnesium, 13 mg/kg green tea extract, 3 mg/kg N-acetyl-cysteine, 0.0004 mg/kg selenium, 0.03 mg/kg copper, 0.01 mg/kg manganese.

The compositions of the present invention may be administered by a variety of routes which include, but are not limited to oral, intravenous, or parenteral administration. Precise dosages for oral, intravenous, or parenteral administration may vary-and will be determined based on experience with the individual subject treated. Preferably, the pharmaceutical composition is in unit dosage form, e.g. as tablets or capsules. In such form, the composition is sub-divided into unit doses containing appropriate quantities of the active ingredient; the unit dosage forms can be packaged compositions, for example, packaged powders, vials, or ampoules. The unit dosage form can be, for example, a capsule, a pill or tablet itself, or it can be the appropriate number of any such compositions in package form.

Another aspect of the present invention is to provide an effective amount of the compositions and a pharmaceutically acceptable carrier, diluent, or excipient.

Another aspect of the present invention is to provide pharmaceutical compositions comprising comprising lysine, proline, arginine, ascorbic acid, magnesium, green tea extract, N-acetyl-cysteine, selenium, copper, and manganese, and optionally an effective amount of estrogen or progestin.

Pharmaceutical formulations of the present invention can be prepared by procedures known in the art using well known and readily available ingredients. For example, the ingredients of the present compositions, with or without an estrogen or progestin compound, can be formulated with common excipients, diluents, or carriers, and formed into tablets, capsules, suspensions, powders, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, and glycerol monostearate; adsorptive carriers such as kaolin and bentonite; and lubricants such as talc, calcium and magnesium stearate, and solid polyethyl glycols.

The therapeutic compounds of the present invention may be formulated into pharmaceutical compositions that may optimize or facilitate their use. In particular, the pharmaceutical compositions contains effective amounts for the treatment of extracellular matrix disorder associated with estrogen replacement. Such pharmaceutical compositions often contain a pharmaceutically acceptable carrier or diluent, and if appropriate, an excipient.

The compositions also can be formulated as elixirs or solutions for convenient oral administration or as solutions appropriate for parenteral administration, for example, by intramuscular, subcutaneous or intravenous routes. Ideally the formulation is in the form of a pill, tablet, capsule, lozenge, liquid or similar dosage form. The compositions may well be suited to formulation as sustained release dosage forms and the like. The formulations can be so constituted that they release the active ingredient only or preferably in a particular physiological location, possibly over a period of time. The coatings, envelopes, and protective matrices may be made, for example, from polymeric substances or waxes.

Pharmaceutical formulations of the present invention can be prepared by procedures known in the art using well known and readily available ingredients. For example, the compounds of formula I, with or without an estrogen or progestin compound, can be formulated with common excipients, diluents, or carriers, and formed into tablets, capsules, suspensions, powders, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl-pyrrolidone; moisturizing agents such as glycerol; disintegrating agents such as calcium carbonate and sodium bicarbonate; agents for retarding dissolutions such as paraffin; resorption accelerators such as quaternary ammonium compounds; surface active agents such as cetyl alcohol, and glycerol monostearate; adsorptive carriers such as kaolin and bentonite; and lubricants such as talc, calcium and magnesium stearate, and solid polyethyl glycols

The compounds also can be formulated as elixirs or solutions for convenient oral administration or as solutions appropriate for parenteral administration, for example, by intramuscular, subcutaneous or intravenous routes. Additionally, the compounds are well suited to formulation as sustained release dosage forms and the like. The formulations can be so constituted that they release the active ingredient only or preferably in a particular physiological location, possibly over a period of time. The coatings, envelopes, and protective matrices may be made, for example, from polymeric substances or waxes.

Unless otherwise defined, all scientific terms used herein have the same meaning as commonly understood by one of ordinary skill in the art. Exemplary methods and materials are described below and their equivalents can be used. All publications and other references mentioned herein are incorporated by reference in their entirety.

The following examples are presented to further illustrate the present invention. It is not intended that the invention be limited in scope by reason of any of the following examples.

Experiments

Experimental Rationale and Protocols

Growth Rate Assay for Smooth Muscle Cells

Rationale: As described, the excessive growth rate of smooth muscle cells and cancer cells is directly related to accelerated atherosclerotic process and malignant tumor growth, respectively. Cultured cell growth rate is estimated according to de novo DNA synthesis assessed (i.e., [³H]Thymidine Incorporation) according to the amount of Tritium-labeled metabolic precursor incorporated into cellular DNA during incubation period.

[³H]Thymidine Incorporation

Human smooth muscle cells (Aortic-Cambrex Corporation, San Diego, Calif., Cat. No. CC-2571) were seeded at a cell concentration of 40,000 cells/ml (0.5 mL) per well in 24-well plates containing an appropriate growth medium (DMEM plus 10% FBS). After three hours incubation, cells were washed and fresh culture media containing tested compositions at indicated amounts were added Cells were allowed to culture for an additional 3-4 days. At the end of the culture, an aliquot of [³H]thymidine (DuPont-NEN, Boston, Mass.) was added at a final concentration of 1 microCi/mL (1 mCi=37 kBq). After 24 hours, cells were washed in ice-cold Dulbecco's PBS, fixed in cold 10% TCA (trichloroacetic acid) for at least 0.5 hour, and incubated with 0.5 mL 0.1N sodium hydroxide for 2 hours at 37° C. Cell bound [³H]thymidine was extracted in 0.1 N NaOH and measured in a liquid scintillation counter (LS 8000, Beckman Coulter).

Smooth Muscle Cell Growth and Invasion as an indicator of Cardiovascular Disease Progession

Rationale: In response to pathological stimuli, smooth muscle cells first migrate from the media layer to the intima layer of the arterial wall, and then proliferate within the intima layer. These events are crucial in the initial development of atherosclerotic plaques. Formation of atherosclerotic lesions in the intima layer occurs in many cardiovascular diseases including hypertension, atherosclerosis, myocardial ischemia, infarction and stroke. (R. Ross, Cellular Mechanisms of Atherosclerosis, Atherosclerosis Review, 103, Vol. 25, pages 195-200). The present compositions are designed to inhibit the invasion and proliferation of smooth muscle cells and is believed to be a remedy alleviating the side-effects of estrogen replacement therapy in post-menopausal women.

The “composition” used in the following experiments refers to a composition containing the following specific ingredients in the specific amounts: lysine is present at 1 gram, proline is present at 750 mg, arginine is present at 500 mg, ascorbic acid is present at 710 mg, magnesium is present at 50 mg, green tea extract is present at 1 gram, N-acetyl-cysteine is present at 200 mg, selenium is present at 30 meg, copper is present at 2 mg, and manganese is present at 1 mg. Capsules containing the above-mentioned composition was first dissolved in culture media and diluted to appropriate concentrations prior to use.

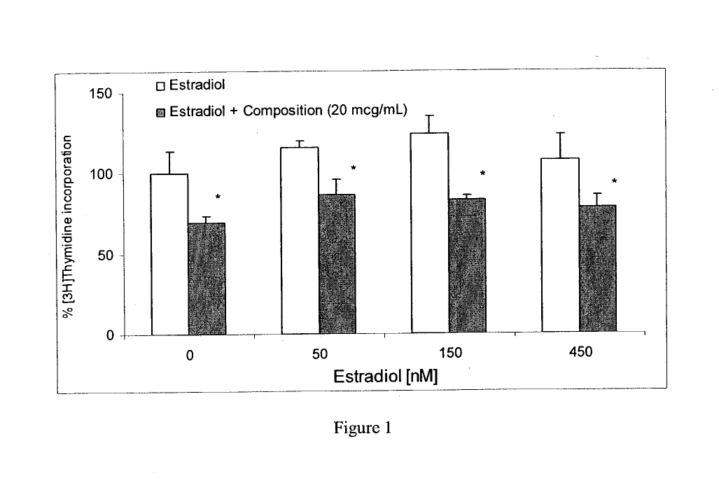

Data represented in FIG. 1 show in vitro experiments on smooth muscle cell proliferation using estrogen alone and estrogen with the composition (containing lysine 1 gram, proline 750 mg, arginine 500 mg, ascorbic acid 710 mg, magnesium 50 mg, green tea extract 1 gram, N-acetyl-cysteine 200 mg, selenium 30 mcg, copper 2 mg, and manganese 1 mg). Results from representative experiments are shown and values represent the (mean.±SEM). Comparisons were subjected to ANOVA followed by Fisher's least significant difference test. Significance was accepted at P<0.05. “%” refers to % of control value; for example % [³H]thymidine incorporation refers to % in reference to control cells.

As shown in FIG. 1, estrogen between the doses of 50-450 nM is found to induce an increase in [³H]thymidine incorporation in human smooth muscle cells, which is in line with the estrogen's association with anti-hypertensive effects. The composition at a concentration of 20 mcg/ml effectively abrogated the estrogen-mediated smooth muscle cell proliferation. The composition was also found to effectively block the [³H]thymidine incorporation mediated by progesterone (50-45 nM) and dehydroepiandrosterone sulfate (25-100 μM). Thus, the composition can effectively block estrogen-induced smooth muscle cell proliferation.

Smooth Muscle Cell Invasion Assay

Elevated capacity of smooth muscle cells and cancer cells to invade extracellular matrix is directly related to the initiation of the atherosclerotic plaque formation and to metastasis formation, respectively. Cultured cell invasiveness is estimated according to the number of cells penetrating through a porous plastic membrane covered with natural extracellular matrix (Matigel, Beckton-Dickinson). Cultured cells were grown in 75 cm² flasks in culture medium containing 10% fetal bovine serum (FBS) to near complete confluency of 37° C. For the last 48 hours of incubation de novo synthesized cellular DNA was metabolically labelled by adding 1 microCi/mL [³H]thymidine. Labelled cells were detached from plastic surface by treating cell layer with 0.025% trypsin in PBS. Cultured cells were seeded on the top surface of the bottom plastic membrane of the inserts placed in the wells of a 24 well plastic plate in 0.5 mL of culture medium containing 10% fetal bovine serum (FBS) at concentration 40,000 cell/mL. Insert bottom membrane has controlled size pores of 8 micron in diameter and is covered with the layer of Matrigel. Cells were allowed to attach to the Matrigel surface for 3 hours by incubation at 37° C. Cell culture medium was replaced with a fresh medium containing no FBS and indicated amounts of tested compounds. Cells were incubated at 37° C. for 24 hours. At the end of incubation period inserts were removed from the wells and washed thoroughly with PBS. Upper surfaces of the insert bottom membrane were wiped with cotton wipe to remove attached cells. Then membranes were cut out and transferred to scintillation vial filled with scintillation fluid. Cell layers were treated with ice-cold 10% TCA for 30 min. Number of cells penetrated to the other side of the porous membrane was assessed according to amount of lower membrane surface-bound radioactivity as measured with scintillation counter (LS 8000, Beckman-Coulter).

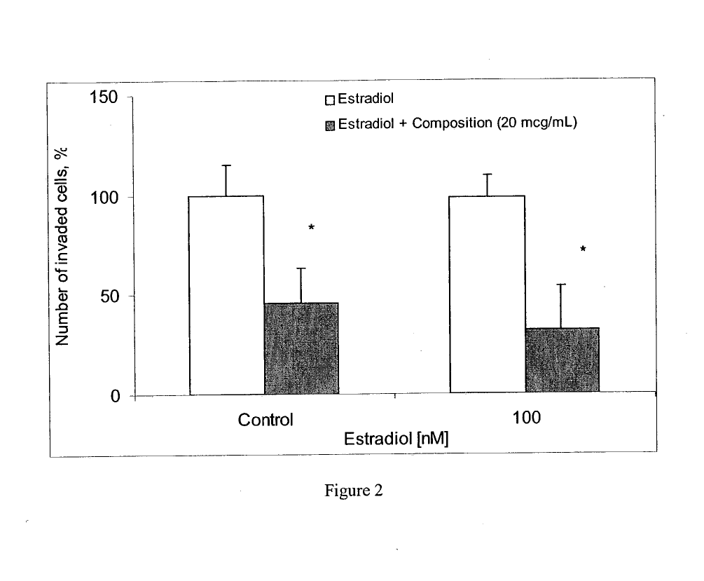

As shown in FIG. 2, estrogen at 100 nM is found to induce an increase in human smooth muscle cell invasion. The composition at a concentration of 20 mcg/ml effectively abrogated the estrogen-mediated smooth muscle cell invasion.

Expression of Interleukine-6 in Smooth Muscle Cells as in Indicator for Autocrine Inflammatory Response

Rationale: Cytokine expression and its involvement in inflammatory responses are known. It has been recently accepted that vascular and smooth muscle pathology manifested in cardiovascular diseases is one of the inflammatory responses during atherosclerosis and hypertension. Interleukin-6 is one of the key cytokines which trigger the inflammation process. Over-expression of interleukin-6 in smooth muscle cells under pathological stimuli may further amplify the inflammatory lesions. The present compositions are designed to inhibit over-expression of cytokine production in smooth muscle cells (in particular, the interleukin-6). By inhibiting the expression of interleukin-6 in smooth muscle cells, the present compositions are believed to be a remedy alleviating the side-effects of estrogen replacement therapy in post-menopausal women.

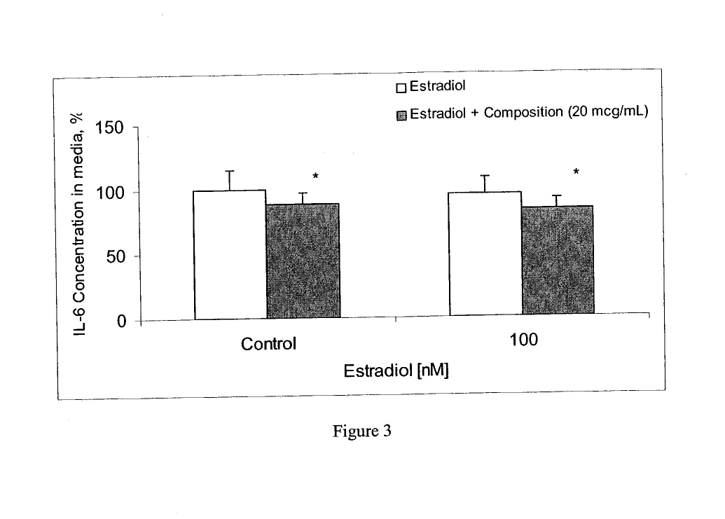

As shown in FIG. 3, estrogen at 100 nM is found to induce an increased in interleukin-6 release in human smooth muscle cells. We found that the composition at a concentration of 20 mcg/ml effectively abrogated the estrogen-mediated release of interleukin-6 in smooth muscle cells.

Breast Cancer Cell Proliferation an Indicator of Neoplastic Disease Progression

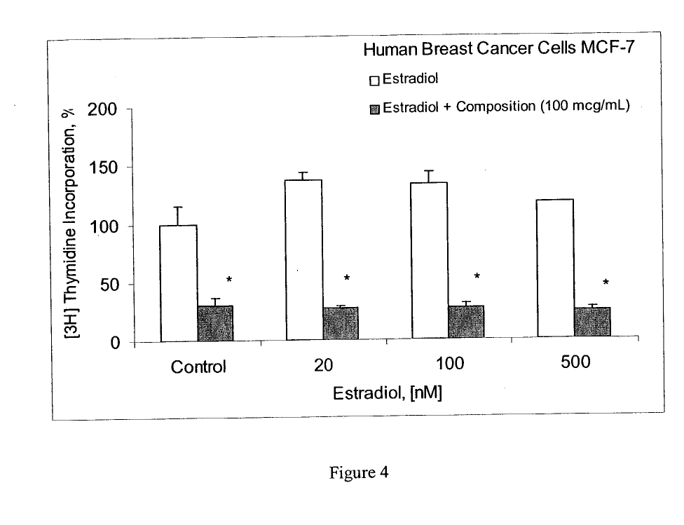

As shown in FIG. 4, estrogen between the doses of 20-500 nM is found to induce an increase in [³H]thymidine incorporation in human breast cancer cells (MCF-7 cells; ATCC No. HTB-22). This observation is consistent with the observation estrgoen therapy in post-menopausal women is associated an increased in incidence of breast cancer. The composition at a concentration of 100 mcg/ml effectively abrogated the estrogen-mediated MCF-7 cell proliferation. Similar inhibition was observed using another breast cell lines (i.e., MDA-MB-MB-231; ATCC No. HTB-26). The composition also found to be effectively block the [³H]thymidine incorporation mediated by progesterone (100 μM). Thus, the composition can effectively block estrogen-induced breast cancer cell proliferation.

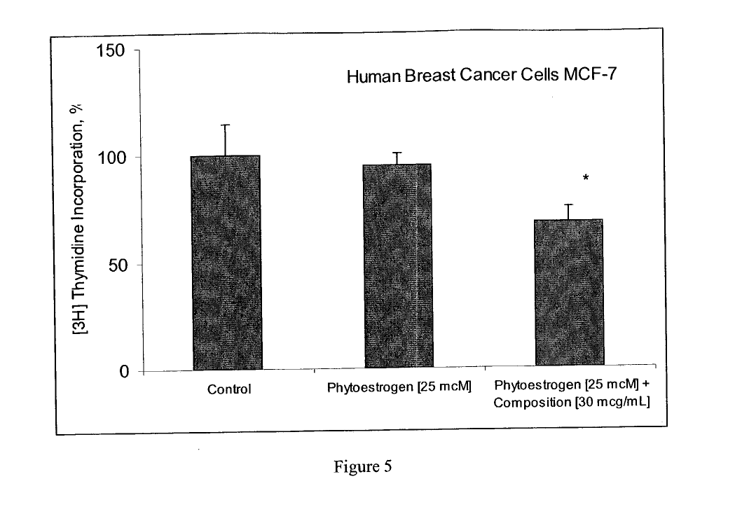

As shown in FIG. 5, the composition at 30 mcg/ml effectively blocked phytoestrogen-mediated (at 25 μM) human breast cell (MCF-7) proliferation as reflected by [³H]thymidine incorporation in these cells.

Measurement of VEGF (Vascular Endothelial Growth Factor) in Cancer Cells Rationale: New vascularization formation (i.e., neo-vascularization) within a tumor bed is essential to the tumor growth. Without the new blood vessel formulation, most tumors can only grow to approximately 1 mm in diameter because the supply of nutrient and oxygen to the growing tumor cells depends on the blood vessels around the tumor. A tumor has the ability to attract new blood vessel formation (neo-vascularization) by releasing a protein known as vascular endothelial growth factor (VEGF). It is desirable to inhibit the VEGF expression in cancer cells so as to prevent neo-vascularization and inhibit tumor growth. (See, Vascular Endothelial Cell Growth Factor (VEGF), an Emerging Target for Cancer Chemotherapy, Shinkaruk, et al., Current Medicinal Chemistry and Anti-Cancer Agents, 2003, Vol. 3, pages 95-117).

Cytokine Expression Assay

The level of cellular vascular endothelium growth factor (VEGF) production is an indicator of a stimulation of neo-vascularization processes. A rate of cytokine synthesis and secretion into medium by culture cells was measured with a commercially available immunochemical assay kit. Cultured cells were seeded in 48 well plates in 0.5 mL of medium containing 10% FBS at concentration 40,000 cell/mL. Cells in the wells were grown to confluent layer by incubation at 37° C. for 2-5 days. Cell layers were washed with phosphate buffered solution (PBS) and fresh culture medium containing tested compounds and no serum was placed to the wells for 24 hours at 37° C. The level of cytokines secreted into cell culture medium was measured using ELISA kits according to manufacturer's protocol (R&D Systems).

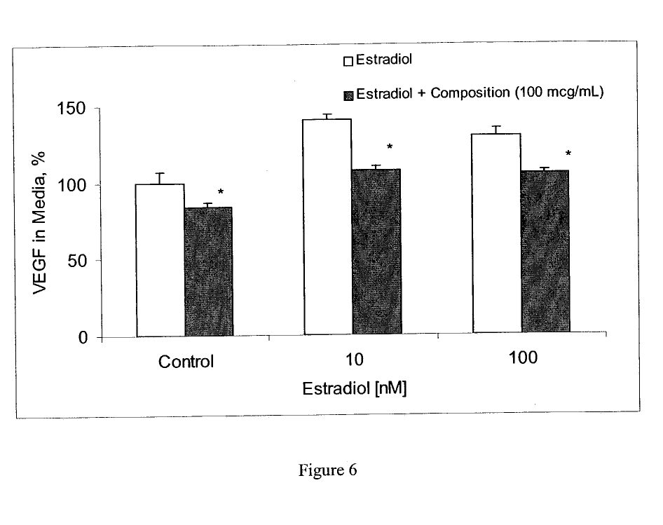

As shown in FIG. 6, estrogen (between 10-100 nM) is found to induce an increased in VEGF release in human breast cancer cells (MCF-7 cells). We found that the composition at a concentration of 100 mcg/ml effectively abrogated the estrogen-mediated release of VEGF in human breast cancer cells. The composition also found to be effectively block the VEGF release in breast cancer cells mediated by progesterone (10 nM). Thus, the composition can effectively block estrogen-induced cytokine-expression in human breast cancer cells.

Clinical Application

The present compositions may be used to counteract estrogen's effect to prevent the degradation of extracellular matrix. The present invention may be used in pathological conditions where side-effects of beneficial hormone therapies are counteracted by the combined use of compositions as disclosed herein. Natural mechanisms to strengthen the connective tissues during and after menopause can be replaced by the therapeutic use of the combinations according to this invention. They are useful to minimize or prevent side-effects of long-term hormone therapies including cancer and other severe health conditions while allowing the desired medical or therapeutic effect of estrogen and related hormones.

Hypertension

Estrogen replacement therapy may exacerbate the atherosclerosis process by affecting, due to its involvement with factors such as fatty substances deposition and fibrosis in the intima of an artery, thickening of the arterial wall. The arterial thickening involves increased intimal smooth muscle cell invasion into the plaque or lesion. If allowed to progress, the arterial wall thickening can cause severe narrowing and obstruction of the lumen of the artery, diminished or occluded blood flow and, consequently, hypertension and ultimately ischemia or infarction of the predominantly affected organ or anatomical part such as the brain, heart, intestine or extremities.

Once the disease has progressed to the stage of significant persistent symptoms and compromised function, artery bypass grafting to replace the damaged artery is necessary. There is a significant risk in artery bypass procedures in the United States. Surgery remains the last option to the solution and morbidity is high. The present invention provides pharmaceutical compositions to alleviate and significantly slow the smooth muscle cell proliferation and migration, hence slowing the process of atherosclerosis and hypertension during estrogen replacement therapy.

Atherosclerosis

Atherosclerosis is associated with cholesterol metabolism which in turn is associated closely with estrogen metabolism. Its rising incidence in women following menopause, and the lower incidence in post-menopausal women receiving estrogen replacement therapy, all point to the moderating influence of estrogens on many aspects including smooth muscle cell migration and cholesterol metabolism. Estrogen is shown to be a potent mitogen and can induce smooth muscle cell migration and proliferation. Another prime effect of estrogens is stimulation of the liver to process cholesterol, particularly the highly atherogenic low-density lipoproteins and very low-density lipoproteins, into bile salts.

It was supposed that when administering estrogen in combination with ascorbate, lysine, proline and other substances the actions of each of the ingredients present in the compositions would cancel each other out. Estrogen treatment leads to degradation of the extracellular matrix while known compositions of ascorbic acids, particularly in combination with lysine and proline, reduce or inhibit such degradations whereby estrogen treatment would become useless. However, surprisingly this is at least partly not the case because of the synergistic effect of such ascorbic acid compositions, particularly when combined with lysine and proline (and lysine at a high amount) which on the one hand prevent or inhibit extracellular matrix degeneration, and on the other hand enhance collagen synthesis, particularly with ascorbic acid which creates and supports the extracellular matrix.

It is known that estrogens and related hormones can weaken the connective tissue. Accordingly, in a preferred embodiment, the present invention provides a combined use or therapeutic application of compounds that counteract the weakening effects of the estrogen compounds. Thus, the present invention provides an ascorbate compound, lysine and proline in an effective amount to strengthen the connective tissue so as to balance the weakening effects by estrogen compounds. Ascorbate is known to stimulate the synthesis of collagen, elastin and other connective tissue macromolecules from fibroblast and related cells. The amino acids lysine and proline are the predominant amino acids required for the synthesis of connective tissue molecules.

In one embodiment, the pharmaceutical compositions of the present invention are shown to be effective in inhibiting smooth cell proliferation. The compositions have clinical relevance in applications such as antihypertensive agents. By reducing smooth muscle cell proliferation, the compositions increase the blood vessel caliber and decrease total peripheral vascular resistance.

In another embodiment, the pharmaceutical compositions of the present invention are shown to inhibit the smooth muscle proliferation that is shown to be essential for the development and progression of atherosclerosis. Our in vitro data show the potent effects of the compositions as inhibitors of proliferation (measured by ³H-thymidine incorporation). It is anticipated that the compositions can thereby attenuate atherosclerosis.

The pharmaceutical compositions of the present invention also inhibit smooth muscle migration and thus attenuate the development and progression of atherosclerosis. Chemotactic migration of medial smooth muscle cells into the intima is an important first step in the pathogenesis of neo-intima formation during atherosclerosis. PDGF is believed to be a key substance for promoting smooth muscle cell migration. (Russel R. (1986) N. Engl. J. Med. 314 488-500). Without being limited by any mechanistic explanation or theory of operation, the ability of the compositions disclosed herewith to inhibit myo-intimal formation in vivo may in part be related to direct inhibition of the physical migration of vascular smooth muscle from the tunic a media into the tunic a intima.

In another embodiment, the present invention provides pharmaceutical compositions comprising lysine, proline, arginine, ascorbic acid, magnesium, green tea extract, N-acetyl-cysteine, selenium, copper, and manganese, and optionally estrogen and progestins, or a pharmaceutically acceptable excipient thereof, for inhibiting proliferation of smooth muscle cells in mammals, preferably human beings, particularly for inhibiting proliferation in blood vessels of post-menopausal women; for inhibiting the development of atherosclerosis; and for suppressing the progression of vascular hypertrophy associated with hypertension.

In another embodiment, the present invention also provides a method of treatment for inhibition of proliferation and migration of smooth muscle cells in mammals, preferably human beings, particularly a method of treatment for preventing proliferation in blood vessels of post-menopausal women, for inhibiting the development of atherosclerosis; or for suppressing the progression of vascular hypertrophy associated with hypertension, said method comprising administering to a patient in need thereof an effective dose of a pharmaceutical composition disclosed herein comprising lysine, proline, arginine, ascorbic acid, magnesium, green tea extract, N-acetyl-cysteine, selenium, copper, and manganese, optionally estrogen and progestins, and a pharmaceutically acceptable excipient thereof. Compositions of the present invention are shown to be effective in inhibiting vascular smooth muscle cell proliferation and migration mediated by a wide variety of different mitogens.

Neoplastic Diseases

There is a strong association between women in certain stages of the menstrual cycle and breast cancer, which suggests that estrogen plays a major role in its pathogenesis. Breast cancer remains a prevalent cancer and clinical studies have shown that approximately one third of breast tumors are estrogen-dependent. This means that estrogens are required for the growth of such breast tumors in both pre-menopausal and post-menopausal patients. In post-menopausal women, in whom breast cancer most commonly occurs, breast tumor concentrations of estrone and estradiol are considerably higher than blood estrogen levels. This observation correlates with the presence of estrogen receptors in breast tumors. Proliferation and metastasis of breast cancer may well be estrogen-dependent. It is believed that women whose breast cancer cells contain estrogen receptors have a much better chance of survival if they are treated with estrogen blocking drugs such as Tamoxifen, a non-steroidal estrogen antagonist.

In another embodiment, the pharmaceutical compositions of the present invention are shown to inhibit the cancer cell proliferation and migration that are essential for the development and progression of breast cancers.

In another embodiment, the present invention provides pharmaceutical compositions comprising lysine, proline, arginine, ascorbic acid, magnesium, green tea extract, N-acetyl-cysteine, selenium, copper, and manganese, optionally estrogen and progestin, or a pharmaceutically acceptable excipient thereof, for inhibiting proliferation of breast cancer cells in mammals, preferably human beings, particularly for inhibiting development of breast cancer.

In another embodiment, the present invention also provides a method of treatment for inhibiting the proliferation and migration of breast cancer cells in mammals, preferably human beings, said method comprising administering to a patient in need thereof an effective dose of a pharmaceutical composition comprising lysine, proline, arginine, ascorbic acid, magnesium, green tea extract, N-acetyl-cysteine, selenium, copper, and manganese, optionally estrogen and progestins, or a pharmaceutically acceptable excipient thereof.

It will be understood that there is no intent to limit the present invention to the preferred embodiment disclosed, but rather it is intended to cover all modifications and alternate constructions falling within the spirit and scope of the invention.

What is claimed is:

1. A pharmaceutical composition for alleviating pathological conditions in a post-menopausal woman, comprising lysine, proline, arginine, ascorbic acid, magnesium, green tea extract, N-acetyl-cysteine, selenium, copper, manganese and one pharmaceutical acceptable component selected from the group consisting of a carrier, a diluent, and an excipient, wherein the composition contains 24-25 wt % lysine, 16-25 wt % ascorbic acid and 22-25 wt % green tea extract.

2. The pharmaceutical composition of claim 1, further comprising an estrogen compound selected from the group consisting of ethynyl estrogen, mestranol, estradiol, ethinyl estradiol, estriol, norethisterone, lynestrenol, ethynodiol, dienoestrol, biperazine estrone sulfate, and phytoestrogen.

3. The pharmaceutical compound of claim 2, further comprising a progestin compound selected from the group consisting of medroxyprogesterone, norethylnodrel, and nonethindrone.

4. The pharmaceutical composition of claim 1, wherein lysine is present at 750 mg-15 gram, proline is present at 500 mg-10 gram, arginine is present at 400 mg-8 gram, ascorbic acid is present at 500 mg-10 gram, magnesium is present at 40 mg-750 mg, green tea extract is present at 750 mg-15 gram, N-acetyl-cysteine is present at 150 mg-2 gram, selenium is present at 20-700 mcg, copper is present at 1.5 mg-20 mg, and manganese is present at 0.8 mg-15 mg.

5. The composition of claim 1, wherein lysine is present at 1 gram-5.5 gram, proline is present at 750 mg-4 gram , arginine is present at 500 mg-3 gram, ascorbic acid is present at 710 mg-4 gram, magnesium is present at 50 mg-300 mg, green tea extract is present at 1 gram-5 gram, N-acetyl-cysteine is present at 200 mg-1 gram, selenium is present at 30-400 mcg, copper is present at 2 mg-10 mg, and manganese is present at 1 mg-8 mg.

6. The composition of claim 1, wherein lysine is present at 1 gram, proline is present at 750 mg, arginine is present at 500 mg, ascorbic acid is present at 710 mg, magnesium is present at 50 mg, green tea extract is present at 1 gram, N-acetyl-cysteine is present at 200 mg, selenium is present at 30 mcg, copper is present at 2 mg, and manganese is present at 1 mg.

7. The pharmaceutical composition of claim 1, wherein the pathological condition is at least one disease selected from the group consisting of hypertension, atherosclerosis and breast cancer.

8. The pharmaceutical composition of claim 1, wherein the composition is in a oral form or a parenteral form.

9. The pharmaceutical composition of claim 8, wherein the oral form is a tablet, a pill or a capsule.

10. A method for alleviating pathological conditions in a post-menopausal woman, comprising the step of administering to the woman in need of treatment an effective amount of the pharmaceutical composition of claim 1, 2 or 3.

11. The method of claim 10, wherein the effective amount of the composition is a daily dosage of 10-208 mg/kg lysine, 7-139 mg/kg proline, 5-111 mg/kg arginine, 7-139 mg/kg ascorbic acid, 0.5-10 mg/kg magnesium, 10-208 mg/kg green tea extract, 2-28 mg/kg N-acetyl-cysteine, 0.0003-0.01 mg/kg selenium, 0.02-0.3 mg/kg copper, 0.01-0.2 mg/kg manganese.

12. The method of claim 10, wherein the effective amount of the composition is a daily dosage of 13-70 mg/kg lysine, 10-56 mg/kg proline, 7-42 mg/kg arginine, 9.8-4 mg/kg ascorbic acid, 0.7-4.2 mg/kg magnesium, 13-70 mg/kg green tea extract, 3-14 mg/kg N-acetyl-cysteine, 0.0004-0.006 mg/kg selenium, 0.03-0.15 mg/kg copper, 0.01-0.1 mg/kg manganese.

13. The method of claim 10, wherein the effective amount of the composition is a daily dosage of 13 mg/kg lysine, 10 mg/kg proline, 7 mg/kg arginine, 56 mg/kg ascorbic acid, 0.7 mg/kg magnesium, 13 mg/kg green tea extract, 3 mg/kg N-acetyl-cysteine, 0.0004 mg/kg selenium, 0.03 mg/kg copper, 0.01 mg/kg manganese.

14. The method of claim 10, wherein the pharmaceutical composition is administered orally, intravenously, or parenterally.
